(19) United States
(12) Patent Application Publication
(10) Pub. No.: US 2025/0302491 A1
(43) Pub. Date: Oct. 2, 2025

(54) GUIDED HELICAL OSTEOTOME BLADE SYSTEM FOR EXPLANTING AN ANATOMICAL HUMERAL STEM

(71) Applicant: Zimmer, Inc., Warsaw, IN (US)

(72) Inventor: William J. Ivan, Woodbridge, NJ (US)

(21) Appl. No.: 19/036,392

(22) Filed: Jan. 24, 2025

Related U.S. Application Data
(60) Provisional application No. 63/570,453, filed on Mar. 27, 2024.

(51) Int. Cl. A61B 17/16

(52) U.S. Cl. CPC A61B 17/1604; A61B 17/162; A61B 17/1664

(57) ABSTRACT

An apparatus can include an osteotome blade including an elongate main body extending from a proximal end to a distal end, the elongate main body having a curved cross-section and defining a helical path along at least a portion of a longitudinal axis of the elongate main body, wherein the distal end includes a distal cutting edge.

### CLAIM OF PRIORITY

This application claims the benefit of U.S. Provisional Patent Application Ser. No. 63/570,453, filed on Mar. 27, 2024, the benefit of priority of which is claimed hereby, and which is incorporated by reference herein in its entirety.

### TECHNICAL FIELD

The present application relates to an orthopedic system and specifically to a shoulder implant revision system.

### BACKGROUND

In a healthy shoulder, the proximal humerus is generally ball-shaped, and articulates within a socket formed by the scapula, called the glenoid, to form the shoulder joint. Some implant systems for the replacement of the shoulder joint generally replicate the natural anatomy of the shoulder. Such implant systems can include a humeral component having a stem that fits within the humeral canal, and an articulating head that articulates within the socket of a glenoid component implanted within the glenoid of the scapula. Reverse-type shoulder implant systems have been developed in which the conventional ball-and-socket configuration that replicates the natural anatomy of the shoulder is reversed, such that a concave recessed articulating component is provided at the proximal end of the humeral component that articulates against a convex portion of a glenosphere of a glenoid component.

Whether standard or reverse, the humeral component of traditional shoulder arthroplasty is traditionally designed with a stemmed implant. Orthopedic implants which comprise porous metallic components, such as Trabecular Metal®, have been successfully used by surgeons for over two decades since the physical and mechanical properties of the porous metal promotes superior biologic fixation to the host bone. However, explanting a well-fixed anatomical humeral stem with a porous proximal section and a smooth distal section during revision shoulder arthroplasty, for example, can present a significant challenge especially since preservation of bone stock is structurally critical for supporting the subsequent placement of a new shoulder prostheses.

### Overview

To further illustrate apparatuses and methods disclosed herein, a non-limiting list of examples is provided here:

Example 1 is an apparatus including an osteotome blade including an elongate main body extending from a proximal end to a distal end, the elongate main body having a curved cross-section and defining a helical path along at least a portion of a longitudinal axis of the elongate main body, wherein the distal end includes a distal cutting edge.

Example 2 is the apparatus of Example 1, wherein optionally the distal end includes a second cutting edge along a side surface of the main body proximate the distal cutting edge of the main body.

Example 3 is the apparatus of any of Examples 1-2, wherein optionally the curved cross-section of the elongate main body defines a partially cylindrical cross-section.

Example 4 is the apparatus of any of Example 1-3, wherein optionally an inside diameter of the main body approximates an outside diameter of a humeral stem.

Example 5 is the apparatus of any of Examples 1-4, wherein optionally a width of the main body tapers down towards the distal end.

Example 6 is the apparatus of any of Examples 1-5, wherein optionally the curved cross-section defines a circular arc.

Example 7 is the apparatus of any of Examples 1-6, wherein optionally the distal cutting edge defines a sharpened edge along an entire width of a distal edge of the main body.

Example 8 is the apparatus of any of Examples 1-7, wherein optionally the helical path is one of a left twisting helix or a right twisting helix.

Example 9 is the apparatus of any of Examples 1-8, wherein optionally a proximal portion of the main body is straight and the helical path begins approximately halfway down the main body and continues to the distal cutting edge.

Example 10 is a system for explanting a humeral stem including an osteotome blade including an elongate main body extending from a proximal end to a distal end, an attachment section at the proximal end to removably attach to a chuck, and a curved cross-section, wherein the elongate body defines a helical path along at least a portion of a longitudinal axis of the elongate main body, wherein the distal end includes a distal cutting edge, a cannulated blade chuck configured to hold the osteotome blade, a blade guide, wherein the blade guide is configured to be removably attached to a humeral stem, the blade guide including a slot having a curved shape to receive the osteotome blade in a sliding configuration, and a guide rod which articulates with the blade guide, wherein the cannulated blade chuck is configured to slide up and down the guide rod to repeatedly drive the osteotome blade back and forth through the slot in the blade guide.

Example 11 is the system of Example 10, wherein optionally the blade chuck includes a body having a central passage and a mounting section for mounting the blade thereto.

Example 12 is the system of any of Examples 10-11, wherein optionally the blade guide includes a movable stem to attach to taper of humeral stem and solid stem to removably engage with a notch on the humeral stem, wherein the movable stem and the solid stem are configured to solidly hold the blade guide in place relative to the humeral stem.

Example 13 is the system of any of Examples 10-12, wherein optionally the slot is angled so as to have a similar angle as a taper of an upper porous portion of the humeral stem.

Example 14 is the system of any of Examples 10-12, wherein optionally the blade guide includes an articulation cavity to receive a bulbous end of the guide rod in an articulating manner to allow the guide rod to articulate relative to the blade guide.

Example 15 is the system of any of Example 10-14, wherein optionally further including a hammer assembly coupleable to the cannulated blade chuck.

Example 16 is a method of explanting a humeral component including repeatedly driving an osteotome blade along an outer surface of a humeral stem, and a distal cutting edge of the osteotome blade twisting around a collar and a fin of the humeral stem due to a shape of the osteotome blade.

Example 17 is the method of Example 16, wherein optionally the osteotome blade includes an elongate main body extending from a proximal end to a distal end, the elongate main body and defining a helical path along at least a portion of a longitudinal axis of the elongate main body, wherein the distal end includes the distal cutting edge.

Example 18 is the method of any of Examples 16-17, wherein optionally the osteotome blade includes a curved cross-section.

Example 19 is the method of any of Examples 16-18, wherein optionally the distal end includes a second cutting edge along a side surface of the main body proximate the distal cutting edge of the main body.

Example 20 is the method of any of Examples 16-19, wherein optionally further including driving a second osteotome blade, having an opposite twist from the osteotome blade, along a second side of the humeral stem.

In Example 21, the apparatuses or methods of any one or any combination of Examples 1-20 can optionally be configured such that all elements or combinations of elements or options recited are available to use or select from.

These and other examples and features of the present apparatuses and methods will be set forth in part in the following Detailed Description. This Overview is intended to provide non-limiting examples of the present subject matter—it is not intended to provide an exclusive or exhaustive explanation. The Detailed Description below is included to provide further information about the present apparatus, systems, and methods.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a perspective view of a humeral stem within a humerus, in accordance with one embodiment.

FIG. 2 shows a perspective view of a system for explanting a humeral stem, in accordance with one embodiment.

FIG. 3 shows another view of the system for explanting a humeral stem, in accordance with one embodiment.

FIG. 4 shows a top perspective view of an osteotome blade, in accordance with one embodiment.

FIG. 5 shows a side perspective view of the osteotome blade, in accordance with one embodiment.

FIG. 6 shows a perspective view of the osteotome blade, in accordance with one embodiment.

FIG. 7 shows an end view of the osteotome blade, in accordance with one embodiment.

FIG. 8A shows a perspective view of an osteotome blade, in accordance with one embodiment.

FIG. 8B shows a perspective view of the osteotome blade, in accordance with one embodiment.

FIG. 9 shows a top perspective view of the osteotome blade and a chuck, in accordance with one embodiment.

FIG. 10 shows a side view of the osteotome blade and the chuck, in accordance with one embodiment.

FIG. 11 shows a top perspective view of a blade guide, in accordance with one embodiment.

FIG. 12 shows a side view of the blade guide, in accordance with one embodiment.

FIG. 13 shows a perspective view of the blade guide and a guide rod, in accordance with one embodiment.

FIG. 14 shows a close view of the blade guide and the guide rod, in accordance with one embodiment.

FIG. 15 shows a side view of the osteotome blade and the chuck attached to a slap hammer, in accordance with one embodiment.

FIG. 16 shows a side perspective view of the osteotome blade in use, in accordance with one embodiment.

FIG. 17 shows a side perspective view of the osteotome blade in use, in accordance with one embodiment.

FIG. 18 shows a method of explanting a humerus stem implant, in accordance with one embodiment.

### DETAILED DESCRIPTION

As used herein, the following directional definitions apply. Anterior and posterior mean nearer the front or nearer the rear of the body, respectively, proximal and distal mean nearer to or further from the root of a structure, respectively, and medial and lateral mean nearer the sagittal plane or further from the sagittal plane, respectively. The sagittal plane is an imaginary vertical plane through the middle of the body that divides the body into right and left halves.

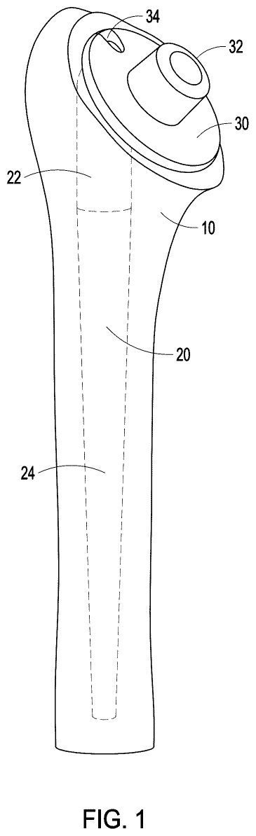

FIG. 1 shows a perspective view of a humeral stem 20 within a humerus 10, in accordance with one embodiment. In this example, the humeral stem 20 includes a porous proximal section 22 and a smooth distal section 24. A collar 30 of the humeral stem 20 can include a tapered connector 32 and a notch 34. The porous section 22 can be formed of porous metallic components, such as Trabecular Metal®.

As noted above, sometimes a revision surgery is necessary and the humeral stem needs to be explanted and removed from the humerus. As an example, one technique for removing a humeral stem implant with a proximal porous section and a smooth distal section describes cutting a window or opening through the cortical wall of the humerus. This technique reduces the amount of porous implant surface area in contact with the humerus to reduce the retrograde stem implant impaction force necessary to drive the stem implant out. Concerns with this technique, however, is that the structural defect (window) that was intentionally created in the humerus may result in localized mechanical stress resulting in a fracture of the host bone either during the revision surgery itself or at a later point in time due to non-healing.

The present system facilitates the explanting of an anatomical humeral stem implant comprised of a proximal porous section and a smooth distal section while minimizing bone loss.

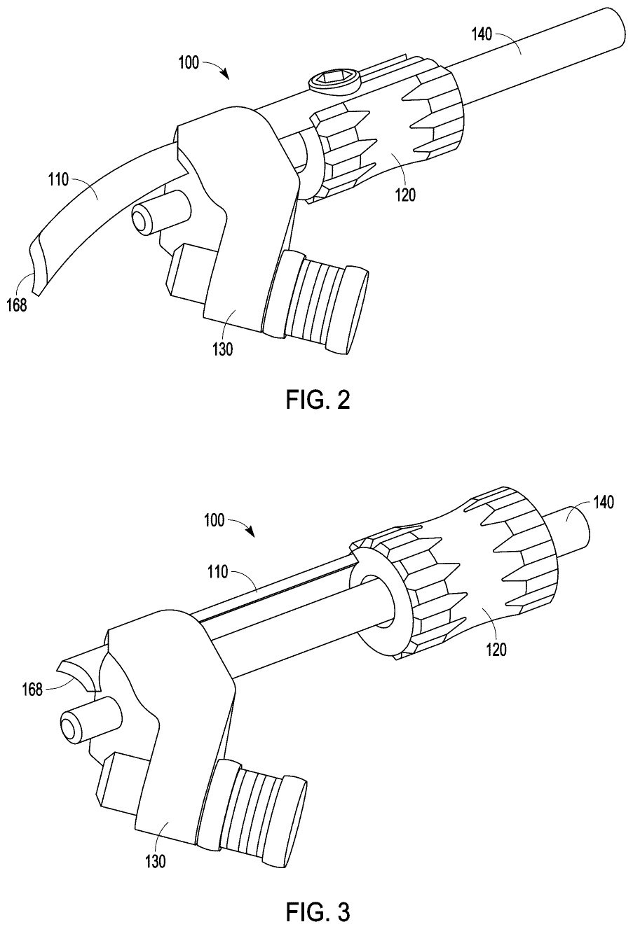

FIG. 2 shows a perspective view of a system 100 for explanting a humeral stem with an osteotome blade 110 advanced, and FIG. 3 shows another view of the system 100 with the blade retracted, in accordance with one embodiment.

In general, the system 100 can include an osteotome blade 110, a cannulated blade chuck 120 configured to hold the osteotome blade 110, a blade guide 130 configured to be removably attached to a humeral stem, and a guide rod 140 which couples to the blade guide 130. Here, the cannulated blade chuck 120 is configured to slide up and down the guide rod 140 so as to repeatedly drive the osteotome blade 110 back and forth through a slot in the blade guide 130 to allow the user to cut away bone material from a humeral stem. The blade 110 is attached to the chuck 120 which translates along the guide rod 140. The blade guide 130 precisely controls the trajectory of the osteotome blade 110.

As will be detailed below, the system is configured to assist in guiding a distal cutting edge 168 of the osteotome blade 110 between the bone and a humeral stem and especially along the surface of a porous section of the humeral stem as the osteotome blade 110 is repeatedly advanced and retracted. The blade 110 is designed to allow for removal of the stem with a minimum of bone loss and without the need to cut a window in the humerus.

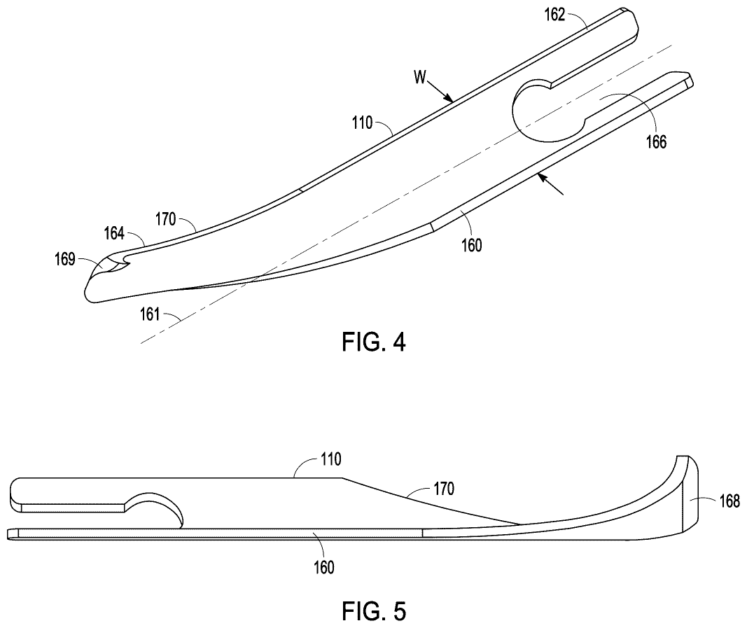

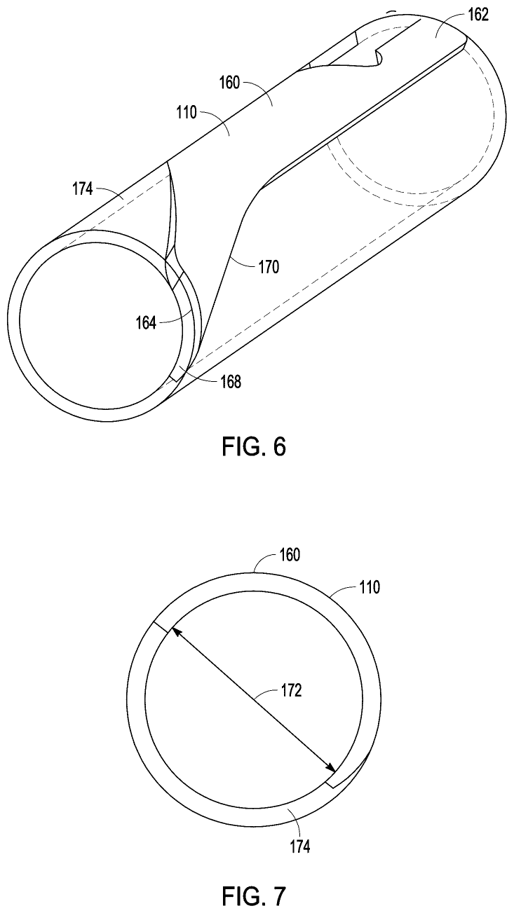

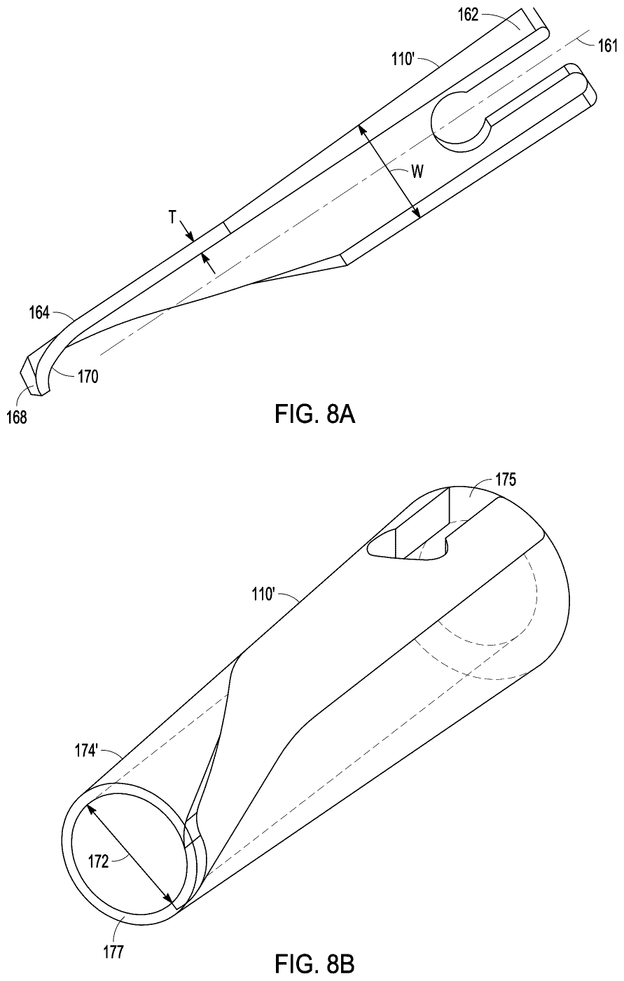

FIGS. 4-7 show the structure and details of the osteotome blade 110. FIG. 4 shows a top perspective view of the osteotome blade 110, FIG. 5 shows a side perspective view of the osteotome blade, FIG. 6 shows a perspective view of the osteotome blade, and FIG. 7 shows an end view of the osteotome blade 110, in accordance with one embodiment.

Here, the osteotome blade 110 can include an elongate main body 160 extending from a proximal end 162 to a distal end 164. The osteotome blade 110 can include an attachment section 166 at the proximal end 162 to removably attach to the chuck 120 (FIG. 2, 3). The blade 110 generally has a curved cross-section and the elongate main body 160 defines a helical path along at least a portion of a longitudinal axis 161 of the elongate main body 160. The distal end 164 can include the sharpened cutting edge 168.

The length of the osteotome blade 110 can be designed so when fully inserted through the blade guide 130 (FIG. 2, 3), the osteotome blade 110 reaches at least to a bottom of the porous section of the humeral stem. (See FIG. 1).

As noted, the elongate main body 160 can have a curved cross-section and define a twisting helical path along at least a portion of a longitudinal axis 161 of the elongate main body 160. As will be described below, the helical twist is designed so that the cutting edge 168 can better reach under a collar and a fin of the humeral stem.

The cutting edge 168 can define a sharpened distal edge along an entire width of the distal edge of the main body 160. In one example, the distal end 164 can include a second sharpened cutting edge 170 along a side surface of the main body 160 proximate the distal cutting edge 168 of the main body.

Referring to FIGS. 6 and 7, the curved cross-section of the elongate main body 160 can define a partially cylindrical cross-section or a partially circular arc cross-section. In one example the cross-section can be hemicylindrical. In a further example, the cross-section can be less than hemicylindrical. Here, an inside diameter 172 of the main body 160 approximates an outside diameter of the tapered porous section 22 of the humeral stem 20 (FIG. 1).

A width W of the main body 160 tapers down towards the distal cutting edge 168, such that the width W at the proximal end 162 is larger than the width W at the distal end 164. This tapering of width W allows for easier insertion through the cutting area of the bone.

Referring to FIG. 6, in an example, the osteotome blade 110 can be manufactured from a cylindrical tube 174. As noted, the inside diameter 172 of the tube 174 can be chosen to have approximately the same diameter as the outside diameter of the humeral stem and specifically the maximum outside diameter of the porous section of the humeral stem. The blade 110 can be machined from the cylindrical tube 174 using conventional machining techniques. The blade 110 can be manufactured from a cylindrical tube 174 with either a left or right helical twist, for example. The helix twist direction is determined using the right-hand rule with the thumb pointing towards the distal end of the blade 110, the remaining fingers curl in the direction of the helix. Thus, FIGS. 4-7 show a left helical twist osteotome blade.

The proximal end 162 portion of the main body 160 can be straight relative to the longitudinal axis and the helical path can begin approximately within the midportion of the main body 160 and continue to the distal cutting edge 168.

Here, the osteotome blade 110 defines a hemicylindrical cross-section with a width that gradually tapers to the distal end 164 while following a helical path about its longitudinal axis. The tapered blade tip can include sharp cutting edges along two edges 168, 170. This allows the blade 110 to cut on both the push and pull stroke. Again, the distal end 164 of the blade includes a narrowed width (relative to the proximal end 162) to minimize the insertion force into the bone.

FIGS. 8A and 8B show another embodiment of an osteotome blade 110′. FIG. 8A shows a perspective view of the osteotome blade 110′ and FIG. 8B shows a perspective view of the osteotome blade 110′, in accordance with one embodiment.

Blade 110′ is similar to blade 110 discussed above, and the above discussion applies herein, with only the main differences pointed out. Here, the osteotome blade 110′ can include an elongate main body extending from a proximal end 162 to a distal end 164. The blade 110′ can generally have a curved cross-section and the elongate main body can define a helical path along at least a portion of a longitudinal axis 161 of the elongate main body. The distal end 164 can include a distal sharpened cutting edge 168 and a side sharpened cutting edge 170.

Here, an inside diameter 172 of blade 110′ approximates an outside diameter of the tapered porous section 22 of the humeral stem 20 (FIG. 1). A width W of the main body tapers down towards the distal cutting edge 168, such that the width W at the proximal end 162 is larger than the width W at the distal end 164. This tapering of width W allows for easier insertion through the cutting area of the bone.

Moreover, the blade 110′ can also have a tapered thickness T. For example, the osteotome blade 110′ can be manufactured from a tapered cylindrical tube 174′. The inside diameter 172 of the tube 174′ can be constant and can be chosen to have approximately the same diameter as the outside diameter of the humeral stem and specifically the maximum outside diameter of the porous section of the humeral stem. In this example, the tube 174′ includes a tapered tube such that a proximal end 175 has a greater outside diameter than a distal end 177. Thus, the thickness T of the blade 110′ will taper down from the proximal end 162 to the distal end 164. This also allows the blade 110′ to be more easily inserted into the bone.

Thus, the osteotome blade 110′ can include a partial cylindrical cross-section that gradually tapers in both width W and thickness T to the distal end 164 while following a helical path about the longitudinal axis 161. The constant inside diameter 172 of the blade 110′ approximates the maximum outside diameter of the tapered porous section of a humeral stem. The tapered blade tip can be provided sharp along two edges 168, 170 and cut on the push and pull stroke. The blade 110′ can be manufactured from a tapered cylindrical tube 174′ having a constant inside diameter in either a left or right helical twist using conventional machine tools, for example.

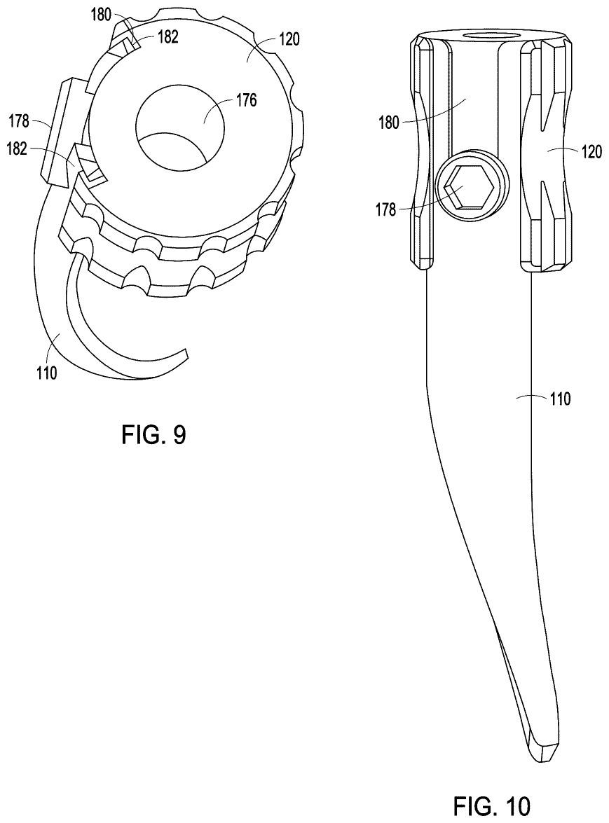

FIGS. 9 and 10 show further details of the osteotome blade 110 attached to the chuck 120. FIG. 9 shows a top perspective view of the osteotome blade 110 and the chuck 120, and FIG. 10 shows a side view of the osteotome blade 110 and the chuck 120, in accordance with one embodiment.

Here, the blade chuck 120 includes a body having a central passage 176 to receive the guide rod 140 and a mounting section 180 for mounting the blade 110 thereto. The mounting section 180 can be a cut-out portion of the chuck 120 and include grooves 182 sized and configured to slidingly receive the blade 110 therein. A fastener 178, such as a screw, can be used to hold the blade 110 in place. The mounting section 180 and grooves 182 are dimensioned to receive both left and right helical twist blades 110.

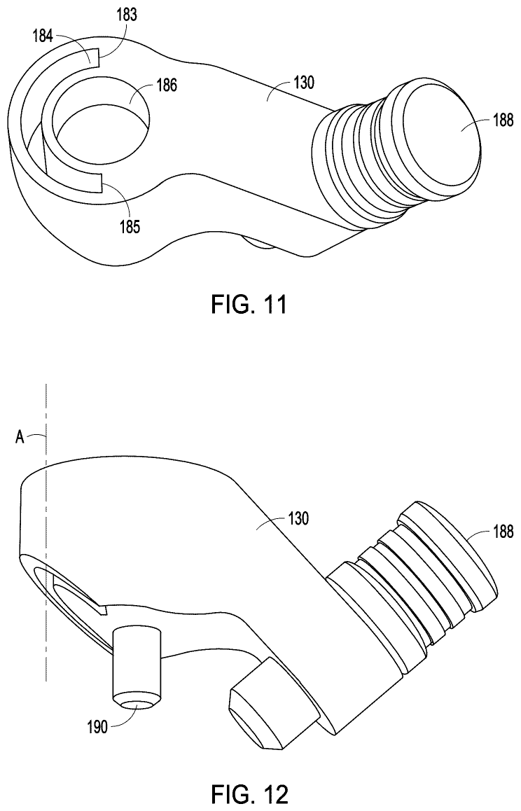

FIG. 11 shows a top perspective view of the blade guide 130, and FIG. 12 shows a side view of the blade guide, in accordance with one embodiment.

In this example, the blade guide 130 can include a movable stem 188 to attach to the taper 32 of the humeral stem (FIG. 1) and solid stem 190 to removably engage with the notch 34 on the humeral stem (FIG. 1). The movable stem 188 is removably mounted through a hole in the guide 130. The movable stem 188 and the solid stem 190 are configured to solidly hold the blade guide 130 in place relative to the humeral stem. Referring also to FIG. 1, when attaching the blade guide 130 to the humeral stem 20, first the solid stem 190 is seated within the notch 34 and then the movable stem is driven through the hole in the blade guide 130 and seated within the taper 32. Further, the blade guide 130 can include an articulation cavity 186 for receiving the guide rod 140, as will be discussed below.

The blade guide 130 includes an arced slot 184 which is curved at a same curvature as the cross-section of the blade 110. This allows the blade 110 to pass back and forth through the slot 184. Thus, the slot 184 is configured to accept and precisely control the trajectory of a blade 110. The slot 184 is angled (from a side view) defining an angle A so as to be similarly angled as the taper of the upper porous section 22 of the humeral stem when the blade guide 130 is mounted to the collar 30 of the humeral stem. The curved slot 184 allows the blade 110 to slide along the slot 184 as the user manipulates the blade so that the bone can be cut away from the outer surface of the porous section of the stem.

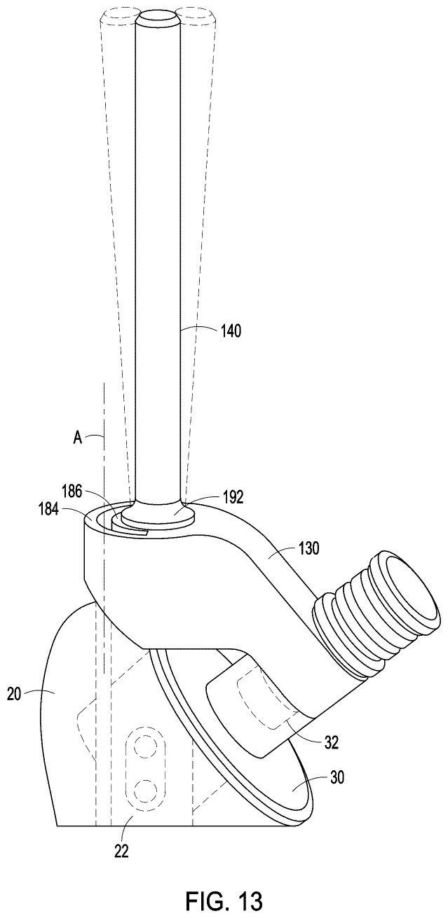

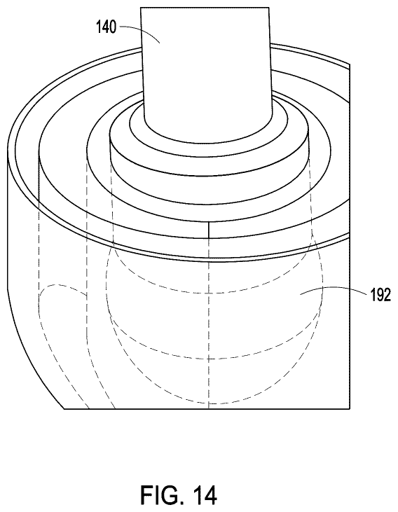

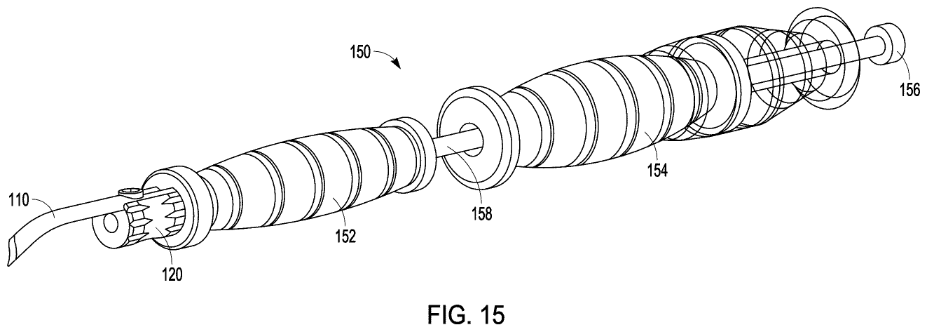

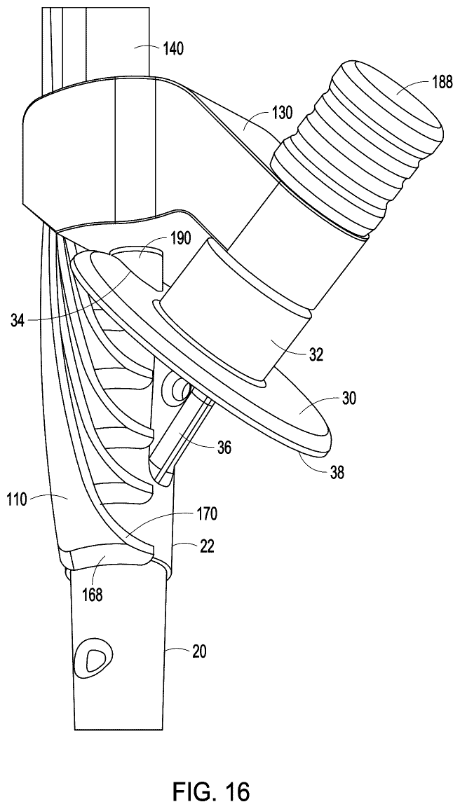

FIG. 13 shows a perspective view of the blade guide 130 and the guide rod 140, and FIG. 14 shows a close view of the blade guide 130 and the guide rod 140, in accordance with one embodiment.

Here, the blade guide articulation cavity 186 includes a spherical pocket dimensioned to receive a bulbous end 192 of the guide rod 140 in an articulating manner to allow the guide rod 140 to articulate relative to the blade guide 130. Thus, as the chuck 120 and blade 110 are translating up and down the guide rod 140, the guide rod 140 can be tilted and moved as needed to prevent the blade 110 from binding within the slot 184 of the blade guide 130.

FIG. 13 further shows how the angle A of the slot 184 of the blade guide 130 defines an angle, when mounted to the collar 30, which matches the angle of the tapered porous section 22 of the humeral stem 20 within the humerus. The articulation of the guide rod 140 is intended to accommodate the angle A of the slot 184 in the blade guide 130 such that the blade guide 130 precisely controls the trajectory of the blade 110 to force the cutting edge of the blade 110 against the outer surface of the porous section 22 of the stem.

FIG. 15 shows a side view the osteotome blade 110 and chuck 120 attached to a slap hammer assembly 150, in accordance with one embodiment.

Here, the hammer assembly 150 can be couplable to the cannulated blade chuck 120. The hammer assembly 150 can include a partially cannulated fixed handle 152 which receives and holds the chuck 120. The hammer assembly 150 further includes a sliding handle 154 which is weighted and rides up and down along a shaft 158 including an end stop 156. As the weight sliding handle 154 is translated up and down the shaft 158 it repeatedly strikes against either the fixed handle 152 or the end stop 156 on the shaft of the to drive the osteotome blade 110 in or out of the humerus respectively.

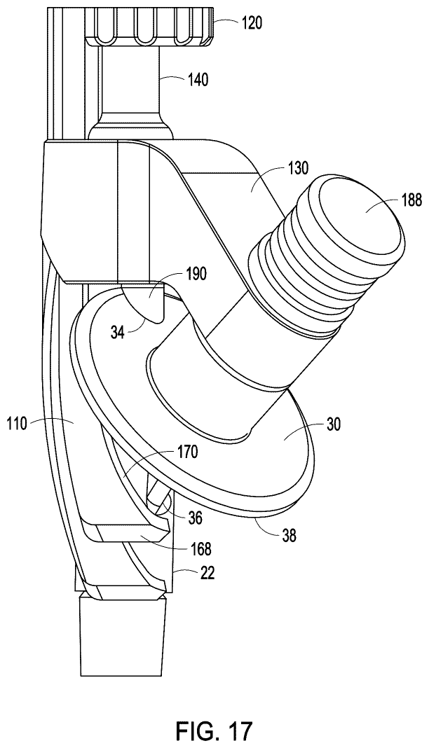

FIG. 16 shows a side perspective view of many successive insertions of the osteotome blade 110 and explant system in use as the blade 110 is repeatedly driven up and down along the implant, in accordance with one embodiment, and FIG. 17 shows a side perspective view of successive insertions of the osteotome blade 110 in use, in accordance with one embodiment.

Here, the blade guide 130 is mounted to the collar 30 of the humeral stem 20 with the movable stem 188 locked within the taper 32 and the stem 190 fixed within the notch 34. As the chuck 120 and blade 110 translate up and down along guide rod 140 (forced by hammer assembly 150, FIG. 15), the user can manipulate the blade 110 such that successive insertions of the blade 110 into the humerus (humerus removed for clarity) and along the outer surface of the porous section 22 of the humeral stem 20 with each insertion cutting deeper. In this example, the user can continually twist the chuck 120 so that the blade 110 moves counterclockwise (from a top view) around the humeral stem 20. As the user continues cutting the bone away from the porous section 22, the helical twist of the osteotome blade 110 allows the cutting edges 168, 170 of the blade 110 to extend under a lower surface 38 of the collar 30 (FIG. 16) and as the user continues to twist the assembly counterclockwise the blade 110 can reach under the fin 36 (FIG. 17). These are the areas which have traditionally been unreachable by conventional osteotome blades.

When the bone on this side of the porous section 22 has been cut away, the user can change out the blade 110 with an opposite helical twist blade, and using the same procedure but twisting the blade clockwise around the stem 20, the user can cut away the bone on the other side of the porous section 22. The stem 20 can then be removed.

Accordingly, the present system allows for explanting an anatomical humeral stem implant comprised of a proximal porous section and a smooth distal section without having to first cut a window or opening through the cortical wall of the humerus. The present system includes a guided helical osteotome blade system that can be used to free the porous proximal section of the anatomical humeral stem implant from the surrounding bone.

The helical osteotome blade can include a partial cylindrical cross-section that gradually tapers to its distal end while following a helical path about its longitudinal axis. The inside diameter of the blade approximates the maximum outside diameter of the tapered porous section of the humeral stem thereby ensuring a close contact between the two when the blade is in use. The cylindrical cross-section of the blade also adds rigidity which allows for a thinner blade to be used for the procedure.

The blade guide 130 forces the blade 110 to maintain close contact between the blade 110 and the surface of the porous section 22 of the humeral stem. Maintaining close contact and utilizing a thin blade both minimize bone loss while explanting the device, which is highly desirable. The narrowed/tapered tip of the blade 110 also reduces its insertion force into the bone. The blade's helical twist allows the cutting edge(s) 168, 170 to reach the entire surface area of the porous section 22 of the humeral stem including the areas under both the fin 36 and under the collar 30 which have traditionally been unreachable by conventional osteotome blades.

The remaining instruments within the system, such as blade guide 130, assist in guiding the distal cutting edge(s) of the osteotome blade between the bone and the humeral stem and especially along the surface of the porous section as the osteotome blade is repeatedly advanced and retracted.

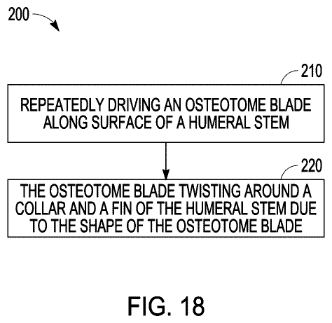

FIG. 18 shows a method (200) of explanting a humerus stem implant, in accordance with one embodiment.

The method (200) can include repeatedly driving an osteotome blade along an outer surface of a humeral stem (210), and a cutting edge of the osteotome blade twisting around a collar and a fin of the humeral stem due to the shape of the osteotome blade (220).

The method can further include removing the first osteotome blade and driving a second osteotome blade, having an opposite helical twist than the first osteotome blade, along a second side of the humeral stem.

In other words, in an example, the user would first use a left twist helical blade to cut out one side of the humeral stem and then take the left twist helical blade off of the chuck and insert a right twist helical blade into the chuck and use the right twist helical blade to cut out the other side of the stem.

Referring to FIGS. 4-8A above, in various examples of the method, the osteotome blade can include an elongate main body extending from a proximal end to a distal end, the elongate main body and defining a helical path along at least a portion of a longitudinal axis of the elongate main body, wherein the distal end includes a distal sharpened cutting edge. The osteotome blade can include a curved cross-section. The distal end can include a second sharpened cutting edge along a side surface of the main body proximate the distal cutting edge of the main body.

In one embodiment, a system can include a kit of parts for explanting a humeral stem. For example, the kit could include a plurality of instruments such as a plurality of left and right helical twist osteotome blades of various lengths and inside diameters, and a plurality of corresponding blade guides which are able to accommodate the full range of porous section diameters and lengths for a given humeral stem implant family.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An osteotome blade comprising:
an elongate main body extending from a proximal end to a distal end;
the elongate main body having a curved cross-section and defining a helical path along at least a portion of a longitudinal axis of the elongate main body;
wherein the distal end includes a distal cutting edge.

2. The osteotome blade of claim 1, wherein the distal end includes a second cutting edge along a side surface of the main body proximate the distal cutting edge of the main body.

3. The osteotome blade of claim 1, wherein the curved cross-section of the elongate main body defines a partially cylindrical cross-section.

4. The osteotome blade of claim 1, wherein an inside diameter of the main body approximates an outside diameter of a humeral stem.

5. The osteotome blade of claim 1, wherein a width of the main body tapers down towards the distal end.

6. The osteotome blade of claim 1, wherein the curved cross-section defines a circular arc.

7. The osteotome blade of claim 1, wherein the distal cutting edge defines a sharpened edge along an entire width of a distal edge of the main body.

8. The osteotome blade of claim 1, wherein the helical path is one of a left twisting helix or a right twisting helix.

9. The osteotome blade of claim 1, wherein a proximal portion of the main body is straight and the helical path begins approximately halfway down the main body and continues to the distal cutting edge.

10. A system for explanting a humeral stem comprising:
an osteotome blade including an elongate main body extending from a proximal end to a distal end, an attachment section at the proximal end to removably attach to a chuck, and a curved cross-section, wherein the elongate body defines a helical path along at least a portion of a longitudinal axis of the elongate main body, wherein the distal end includes a distal cutting edge;
a cannulated blade chuck configured to hold the osteotome blade;
a blade guide, wherein the blade guide is configured to be removably attached to a humeral stem, the blade guide including a slot having a curved shape to receive the osteotome blade in a sliding configuration; and
a guide rod which articulates with the blade guide, wherein the cannulated blade chuck is configured to slide up and down the guide rod to repeatedly drive the osteotome blade back and forth through the slot in the blade guide.

11. The system of claim 10, wherein the blade chuck includes a body having a central passage and a mounting section for mounting the blade thereto.

12. The system of claim 10, wherein the blade guide includes a movable stem to attach to taper of humeral stem and solid stem to removably engage with a notch on the humeral stem, wherein the movable stem and the solid stem are configured to solidly hold the blade guide in place relative to the humeral stem.

13. The system of claim 10, wherein the slot is angled so as to have a similar angle as a taper of an upper porous portion of the humeral stem.

14. The system of claim 10, wherein the blade guide includes an articulation cavity to receive a bulbous end of the guide rod in an articulating manner to allow the guide rod to articulate relative to the blade guide.

15. The system of claim 10, further including a hammer assembly coupleable to the cannulated blade chuck.

16. A method of explanting a humeral component, the method comprising:
repeatedly driving an osteotome blade along an outer surface of a humeral stem; and
a distal cutting edge of the osteotome blade twisting around a collar and a fin of the humeral stem due to a shape of the osteotome blade.

17. The method of claim 16, wherein the osteotome blade includes an elongate main body extending from a proximal end to a distal end, the elongate main body and defining a helical path along at least a portion of a longitudinal axis of the elongate main body, wherein the distal end includes the distal cutting edge.

18. The method of claim 17, wherein the osteotome blade includes a curved cross-section.

19. The method of claim 17, wherein the distal end includes a second cutting edge along a side surface of the main body proximate the distal cutting edge of the main body.

20. The method of claim 17, further including driving a second osteotome blade, having an opposite twist from the osteotome blade, along a second side of the humeral stem.
